(19) United States
(12) Patent Application Publication
(10) Pub. No.: US 2009/0123552 A1
(43) Pub. Date: May 14, 2009

(54) SPHEROIDS PREPARED FROM AN ISOLATED ACTIVE PRINCIPLE OF VEGETABLE ORIGIN AND A SOLUTION OF VEGETABLE ORIGIN CONTAINING THE ACTIVE PRINCIPLE OR A PRECURSOR THEREOF

(76) Inventor: Michel Iderne, Strasbourg (FR)

Correspondence Address: DAVIS & BUJOLD, P.L.L.C., 112 PLEASANT STREET, CONCORD, NH, 03301, US

(21) Appl. No.: 11/816,945

(22) PCT Filed: Feb. 23, 2006
(86) PCT No.: PCT/FR06/00409

§ 371 Date: Dec. 29, 2007

(30) Foreign Application Priority Data
Feb. 24, 2005 (FR) 0501907

(51) Int. Cl. A61K 36/00; A61K 36/282; A61K 31/60; A61K 9/50

(52) U.S. Cl. 424/490; 424/725; 424/740; 514/165

(57) ABSTRACT

The invention relates to spheroids which are obtained using a method comprising the following steps consisting in: supplying an active principle of vegetable origin in an isolated form; supplying a solution of vegetable origin, containing the active principle or a precursor thereof; supplying an absorbing and adsorbing substance having plastic properties that are compatible with the method; mixing said different products and wetting the mixture with a wetting liquid; extruding the wet mixture; and forming spheroids through the spheronization of the extruded product. The invention also relates to sample applications such as artesunate-based antimalarial spheroids and aspring-based spheroids.

This application is a National Stage completion of PCT/FR2006/000409 filed Feb. 23, 2006 which claims priority from French Patent Application Serial No. 05/01907 filed Feb. 24, 2005.

### FIELD OF THE INVENTION

The present invention concerns medicinal compositions in the form of spheroids prepared from both an active principle of vegetable origin in an isolated form and a solution of vegetable origin containing the active principle or a precursor thereof.

It further concerns the method of preparing these spheroids by mixing an active principle of vegetable origin in an isolated form and a solution of vegetable origin containing the active principle or a precursor thereof, with an absorbent substance, followed by extrusion and spheronization.

Finally, it concerns the applications deriving therefrom for the oral administration of these spheroids, preferably in the health domain, but also in the domains of hygiene, dietetics, cosmetology, nutrition, agriculture and so forth, either human or animal.

### BACKGROUND OF THE INVENTION

One goal of the invention is to furnish spheroids supporting pharmacologically activated substances of vegetable origin and to improve effectiveness due to synergy and the fact that they are more easily released within the digestive tract.

The main goal of the invention is to furnish spheroids that have undergone less titration with the active principle than the other conventional dry galenic forms in which this active principle is found alone in isolated form, while maintaining a comparable or even superior degree of effectiveness.

The invention also enables production of spheroids that are equal in effectiveness to the other conventional dry galenic forms, but of reduced toxicity and lower cost.

Yet another goal of the present invention is to supply effective compositions in a much more practical galenic form for patients, and which additionally is much more stable over time, even under unfavorable storage conditions.

Western pharmacology recognizes the therapeutic action of numerous compounds originating from plants.

When a vegetable substance interesting from the medicinal perspective is discovered, the modern Western practice consists of identifying the active principle responsible for the therapeutic effect sought among the multitude of components that can be extracted from this plant, then isolating it in order to use it alone in a specific medication.

This active principle, purified and stripped of any annexed compounds within the plant considered to be useless, is then supposed to produce maximum therapeutic activity. It is then mixed with one or more appropriate excipients to form the medication.

Once identified, the active principle may continue to be extracted and isolated from the vegetable in order to proceed with manufacturing the medication. But most often, it is obtained chemically by hemisynthesis or total synthesis, which ensures a steady supply and constant quality.

In numerous cases, the compound issuing directly from the plant is not stable or does not possess all the required qualities, especially with respect to solubility, for example, to be useful in that state when producing the medicament. This compound is then generally transformed by chemical means to produce a suitable derivative of the precursor vegetable compound, which is then used as the active principle in the final medicament.

The therapeutic activity and thus the efficacy of a medicament formulated according to the principles described above are obviously proportionate to the quantity of active principle present in the medicament.

Unfortunately, the toxicity of the medicament to the organism, intrinsically connected to its activity, increases along with the quantity of active principle absorbed and is responsible for bothersome, undesirable secondary effects which may even pose a health risk.

The recommended active dose of the principle will be defined as being an acceptable compromise between the desired effectiveness, on the one hand, and on the other hand, the risk and danger posed by secondary effects that it may produce.

Therefore, a great need exists for medicinal formulations which, while retaining a satisfactory degree of effectiveness, would be less toxic to the organism and generate fewer secondary effects.

The invention responds to this need in a particularly advantageous way by furnishing medicinal formulations which surprisingly, despite having smaller amounts of the active principles and thus being less toxic to the organism, have a satisfactory or even superior degree of effectiveness.

Furthermore, besides the problem of toxicity, most of the medicaments are relatively high-priced. While the high cost may be acceptable in rich developed countries, it constitutes an insurmountable obstacle in poor third-world countries, depriving a major portion of the population in these countries of vital access to medical care.

This problem becomes even more crucial in the treatment of pathologies that almost exclusively affect third-world countries. This is the case with malaria, for example, which accounts for 400 million clinical cases a year in Africa and which if untreated inevitably leads to death by hemorrhaging.

Quinine, which has cured this sickness for nearly 100 years, is no longer fully effective today and must be replaced by new anti-malarial products. Promising compounds have been extracted from the plant named Artemisia Annua which, after chemical transformation, yield stable molecules such as artemisinine, dihydroartemisinine or arteseunate which can be effectively used as active anti-malarial principles.

However, after becoming interested in these new active principles, the large pharmaceutical companies have abandoned the market. In practice, for their investments to be profitable, the selling price of a conventional medicament obtained from these compounds would have to be higher than the maximum price affordable by the populations this parasite affects.

By decreasing the quantity of the active principle necessary to produce an effective formulation, the invention allows effective medicaments to be produced from Artemisia Annua at a substantially lower cost than conventional medicaments. The invention is therefore responsive to this humanitarian problem and offers hope to all populations concerned.

Additionally, in the countries concerned, the medicaments must tolerate difficult climatic conditions and extremely unfavorable storage conditions. For these reasons, most of the conventional medicaments have a severely reduced storage life under such conditions.

Advantageously, the spheroids according to the invention constitute a highly stable galenic form requiring no particular care during storage and are very tolerant of difficult climatic conditions.

Moreover, this galenic form is extremely practical for patients, as it does not require trained personnel to administer it, unlike conventional injectable medications.

### SUMMARY OF THE INVENTION

The general inventive idea consists of making medicinal spheroids from two sources of an active principle of vegetable origin, on the one hand, an active principle of vegetable origin in an isolated form and on the other hand, a solution of vegetable origin containing the active principle or a precursor thereof. These two products are mixed with an absorbent and adsorbent substance. The mixture is next extruded, and then made into spheroids, giving it a microspheroid physical shape also known as spherules or spheroids.

The invention thus furnishes a method for producing spheroids by mixing a solution of vegetable origin with an absorbent and adsorbent substance having plastic properties that are compatible with the method, wetting the mixture with a wetting liquid if necessary, extruding the wet mixture, and spheronizing the extruded product to form spheroids.

According to the invention, the mixing operation comprises the following steps:

first, furnishing an active principle of vegetable origin in an isolated form;
second, furnishing a solution of vegetable origin containing the active principle or a precursor thereof;
furnishing an absorbent and adsorbent substance possessing plastic properties that are compatible with the method; and
mixing these different products and if necessary, wetting the mixture with a wetting liquid.

It also furnishes spheroids which contain at least on the one hand, an isolated active principle of vegetable origin and on the other hand, a solution of vegetable origin containing the active principle or a precursor thereof absorbed and/or adsorbed on an absorbent and/or adsorbent substance.

Finally, it teaches the use of medicament compositions comprising an active principle of vegetable origin presented in the galenic form of spheroids obtained using the method of the invention.

Obviously, the invention is not limited to the application of treating malaria, even if that is its fundamental use. It may advantageously be applied to numerous other active principles of plant origin for treating numerous other health or other problems.

### BRIEF DESCRIPTION OF THE DRAWINGS

Other characteristics and advantages of the invention will be apparent from reading the following detailed description, taken in conjunction with the attached drawings, in which:

FIG. 1 is a simplified schematic chart of the essential steps of the method according to the invention;

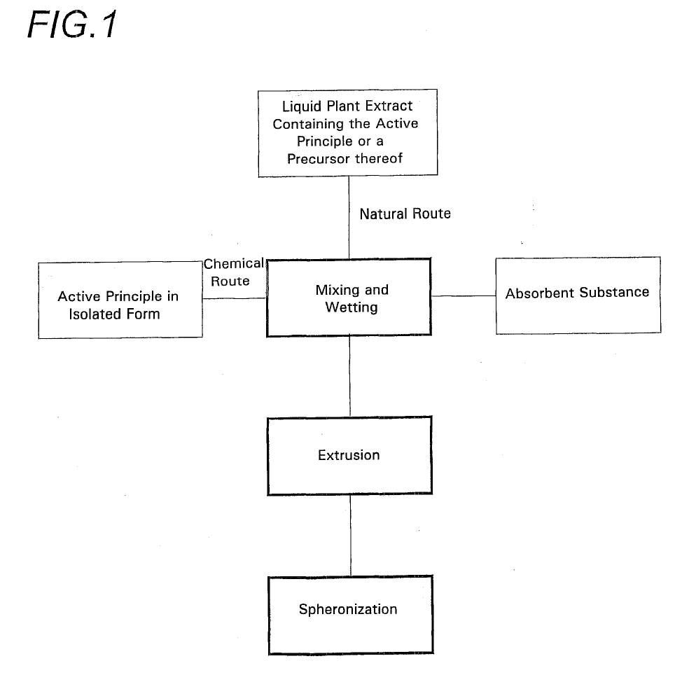

FIG. 2 is a more detailed schematic chart of a first preferred embodiment of the method according to the invention;

FIG. 3 is a schematic chart of a second embodiment of the method according to the invention with the addition of an auxiliary substance or a second active principle during extrusion;

FIG. 4 is a schematic chart of a third embodiment of the method according to the invention with the addition of a second active principle by coating;

FIG. 5 is a schematic chart of a fourth embodiment of the method according to the invention combining the second and third variations;

FIG. 6 is a schematic chart illustrating an application of the method according to the invention for making an anti-malarial composition according to the invention using Artemisia Annua; and

FIG. 7 is a schematic chart illustrating an application of the method according to the invention for making an aspirin-based composition according to the invention.

### DETAILED DESCRIPTION OF THE PREFERRED EMBODIMENTS

As shown in the various drawings, the spheroids according to the invention are made using two sources of an active principle of vegetable origin.

The first of these sources corresponds to the incorporation of an isolated active principle obtained in the conventional manner. This active principal has originated from a vegetable, but may have been chemically transformed and may no longer be identical to the molecule originating directly from the plant. In this way it is possible, for example, to obtain a more stable molecule or one with better adapted properties, specifically higher solubility, or the like.

This active principle may be extracted from the vegetable by any suitable known or as yet undiscovered means. It may also be obtained by chemical means, by total or partial synthesis.

This active principle may take any physical form, specifically, solid, liquid, paste, or gel. Most often at ambient pressure and temperature it is in powder form.

The active principle is not necessarily the major constituent or the most characteristic or well-known constituent of the plant. It may be any compound originating from a plant among the numerous compounds that can be extracted from it.

In this application, active principle means any compound producing an effect that leads to, participates in, or favors the total result produced by the composition, whether it be therapeutic or otherwise.

In order to implement the method of the invention, this active principle may advantageously be used in its isolated commercial form.

This first source of active principle has been symbolically identified as “the chemical route” in the various drawings.

The second source, of the invention, is an active solution of vegetable origin containing the same active principle or a precursor thereof. It is preferably a liquid vegetable extract from the plant from which can be extracted the relevant active principle or its precursor in the case of a chemically transformed active principle.

This active solution of vegetable origin can be obtained from the vegetable by any suitable method, particularly by extraction, preferably aqueous, alcoholic or hydro-alcoholic; by maceration, infusion, decoction, digestion, lixiviation, or the like.

Most often it concerns a liquid vegetable extract and, for example, depending upon the concentration, a mother tincture, an officinal tincture, or a liquid extract. Preferably a mother tincture is used in a small amount, therefore better from an economic standpoint but nevertheless satisfactory for the invention.

However, a dry extract can also be used, having been previously placed in solution or in suspension or used in combination with a wetting liquid in the first step of the invention.

In the drawings, this second enrichment method has been symbolically termed the “natural route.”

The first step of the method according to the invention consists of mixing the products resulting from these two enrichment processes, that is, the isolated active principle, on the one hand, and the active solution of vegetable origin on the other hand, with an absorbent and adsorbent substance.

The absorbent and adsorbent substance is preferably a natural or synthetic polymer type of substance and is most often in powder form. To be acceptable, this absorbent substance must additionally possess plastic properties that are compatible with the subsequent extrusion and spheroid formation steps of the inventive method.

The following are cited as polymers with suitable absorbent and adsorbent properties and satisfactory plastic properties: microcrystalline celluloses, microfine celluloses, starches, modified starches, and polysaccharides.

Also cited is a technical excipient formed of a dry homogeneous mixture of hydroxypropylated cellulose polymer slightly substituted, specifically hydroxypropylated cellulose ether slightly substituted at the level of the β-O-glucopyranosile core groups. This absorbent substance is of particular interest when it is desirable to obtain rapidly dispersing or more strongly titrated spheroids of active principles.

However, it is possible to achieve the invention using any other solid substance with suitable absorbent and adsorbent properties and plastic properties that are compatible with the method of the invention.

In order to endow it with supplemental properties, auxiliary substances may also be added to the mixture, such as, for example, other excipients currently used with existing techniques, specifically binders, sliding agents, lubricating or surface active compounds, or others.

These auxiliary substances may also be compounds designed to improve the results or the quality of the spheroids according to the invention or endow them with new properties. They may consist of vitamins, minerals, and/or other organic components. Some examples to note are simple sugars with low energy power, mineral and organic substances which may correspond, in particular, to lactose, sorbitol, mannitol, maltodextrines, carbonates, citrates, gelatin, polyvinylpyrrolidone or a cellulose derivative (ethyl, methyl, or carboxyl) and in particular, reticulated sodic carboxymethylcellulose.

All of these substances may be mixed simultaneously or successively in any order during the mixing step.

During this mixing step, the mass of dry absorbent substance, for example, microcrystalline cellulose, is wetted if necessary with a wetting liquid until a homogeneous malleable paste is obtained that can withstand the next steps of the procedure, that is, extrusion and spheroid formation.

The solution of vegetable origin produced using the natural route may advantageously serve as the wetting liquid for the wetting process. The addition of another liquid, aqueous or non-aqueous, may sometimes be necessary, however, if an insufficient quantity of liquid is supplied by the vegetable origin solution or, for example, in the case of a vegetable extract in dry form.

The wetting liquid also serves as a vehicle for transporting and depositing the active substances into the core of the absorbent and adsorbent substance in the microcavities of the polymer.

The fabrication process next consists of extruding the wet mass through a die with calibrated orifices, then making the extruded product spherical (spheronization).

During the extrusion process, the wet mass is packed and stretched to transform it into compact filaments of defined, generally cylindrical section called “extrudates.”

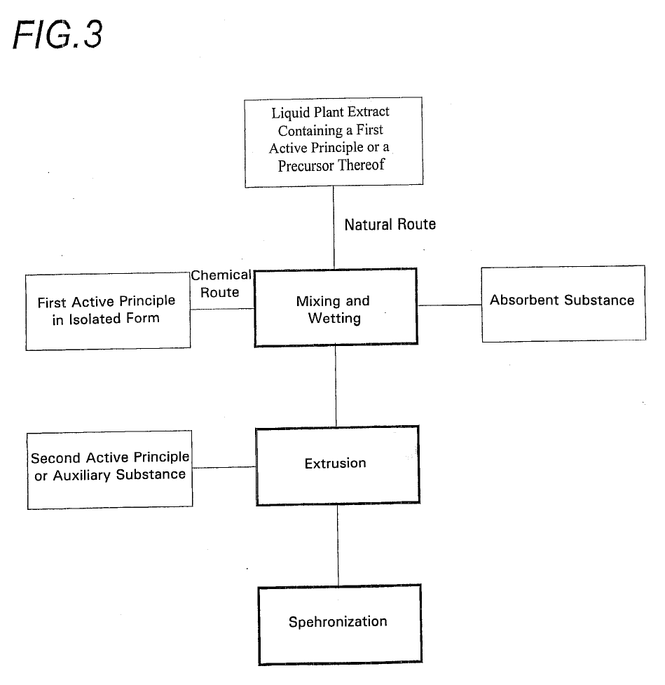

According to a variation of the method shown in FIG. 3, it is possible to introduce during the extrusion step one or more auxiliary substances and/or several other active principles.

This active principle, this auxiliary substance, or this mixture may be incorporated into the “extrudates” by co-extrusion. To do this, it must have plastic properties that are compatible with the co-extrusion process. For this purpose, it may undergo any prior preparatory step. Specifically, it may be mixed with a wetting liquid and/or a suitable adsorbent/absorbent substance.

In order to obtain spheroids, the “extrudates” are placed in a cylindrical apparatus called the “spheronizer” containing a disc in the lower portion spinning at a variable and controlled speed. Due to the effects of centrifugal force exerted by the rotation of the spinning disc, the “extrudates” become regularly fragmented and are then transformed into spheres by a rolling-binding effect.

It has already been stated that the possibility of transforming the extrudates into spheroids of homogeneous sphericity, of previously defined regular granular size, depends as much upon the plastic properties of the extrudates and thus the plastic properties of the wetted mass as it does upon the characteristics associated with the spheronization operation itself, that is, the rotational speed of the spinning disc and the duration of rotation.

To obtain a suitable plasticity that is compatible with the extrusion and subsequent spheroid formation techniques, the mass of the wetting liquid preferably ranges from 3 to 5 times the mass of the absorbent substance used.

A particular feature of the invention cited by way of example consists of preparing spheroids having a granular size ranging from 350 microns (13.8 mil) to 1250 (49.2 mil) microns for an output of at least 95% of the mass of spheroids produced.

In this case, the die of the extruder comprises orifices with openings of 1000 microns (39.4 mils) in diameter and 1000 microns (39.4 mils) long (thickness of the die). The extrusion speed is 100 rpm for a twin screw frontal or other type of extruder.

The spheronization cycle for a 25 cm (9.84 in) diameter or other spheronization apparatus is 5 minutes long with a rotation speed of 1040 rpm.

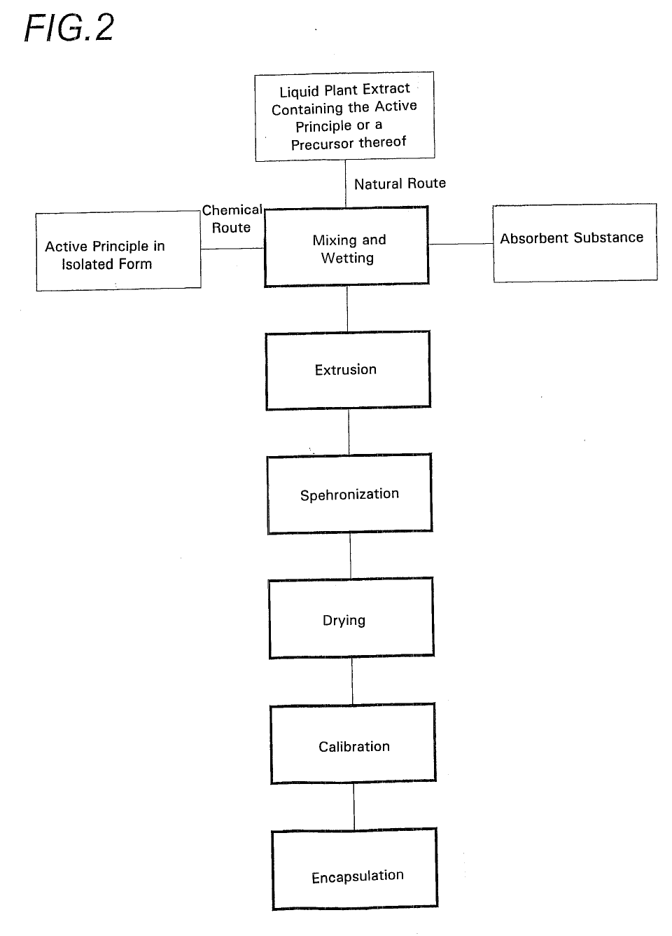

According to the preferred mode of embodiment shown in FIG. 2, the spheroids may then undergo a drying step, preferably at a temperature of about 35-40° C. (95°-104° F.). Otherwise, they may be simply left in the open air for a certain time.

According to the variation in FIG. 2, they may then be passed through a calibration device, which may consists of a unit of sieves, in order to obtain multi-particulate spherical systems of regulated granular size.

The spheroids obtained are stable over time, easy to control, and reproducible. They are simple and practical to use. They can be stored without taking any particular precautions or care and are easily transported because they are light, compact, and not especially fragile.

The spheroids of the present invention may be offered as is or packaged in bulk, in measured distribution units, in capsules, pills, tablets, in packets, or any other suitable form.

They are preferably grouped in capsules corresponding to a predetermined quantity of active principle as a function of the specific dosage of the composition and making a unit dose easier.

Before being packaged in the form of capsules, for example, these multi-particulate systems may be further coated with film during an optional supplemental step of the method according to the invention.

This operation consists of covering them with a resistant film coating that protects the molecules of the active principle, for example, sensitive to the low pH of the stomach or degraded by the enzymes of the intestines.

It also regulates the release of active principle inside the organism. Release can be slowed down in order to selectively deliver the active principle to certain portions of the digestive system, for example, the gastric portion, and in the upper portions of the small intestine.

By using different types of coatings for spheroids within the same capsule, it is also possible to successively deliver several doses of active principle using a single dose of the composition.

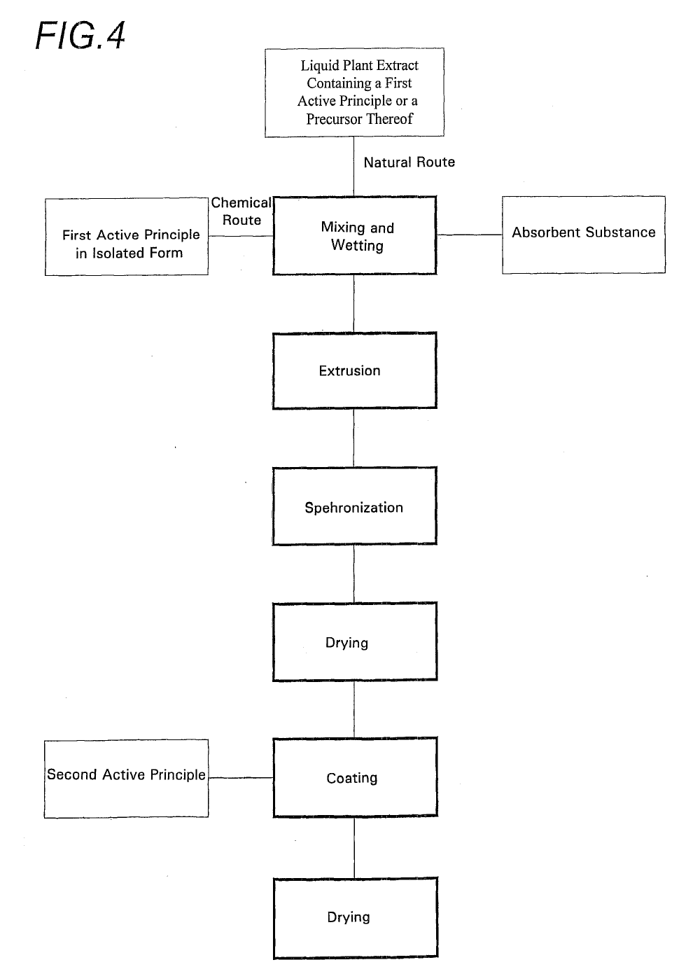

According to another variation of the invention shown more specifically in FIG. 4, the spheroids may also undergo a supplemental coating step that consists of forming a supplemental layer around the spheroids, once they have been formed by the mixing, extrusion, and spheronization steps described above.

The supplemental layer of coating may be composed of one or more auxiliary substances of any type and even preferably of a second active principle, or a mixture containing at least one second active principle different from the one furnished in the initial mixing step that is absorbed and adsorbed into the core of the spheroids.

This second active principle is any type of active substance that is useful for the application considered. It may, for example, act to reinforce or complete the action of the first active principle. It may also perform a different activity, preparing the action of the first active principle, acting on another aspect of the problem to be treated, or treating a secondary effect of the first active principle.

This coating step is preferably carried out after drying the spheroids formed in the spheronization step.

The layer of coating is thus deposited on the spheroids essentially without being mixed with the first active principle present in the spheroid core. This advantageously avoids any problems of interaction and physical-chemical incompatibility between the two active principles. They may co-exist in parallel without harmful interactions, from spheroid production until administration and even over lengthy storage periods.

Therefore, the invention very simply and practically resolves the problem of incompatibility between certain substances that makes their association impossible inside conventional galenic forms, specifically tablets, and which traditionally leads to implementing heavy and/or delicate treatments on the basis of multiple doses or mixing in situ.

The coating step can also produce a delay or a “double discharge” effect between the two active principles, that is, release at different times or in different locations within the organism.

This coating step may be repeated several times so as to form as many successive layers as desired, using different active principles each time or alternating between a certain number of them.

In order to prevent mixing and interaction between the successive layers, it is preferable to interpose a drying step between each coating step.

Thus, the invention also produces spheroids covered with at least one layer of coating containing at least one second active principle different from the one that is absorbed and/or adsorbed onto the absorbent and/or adsorbent substance.

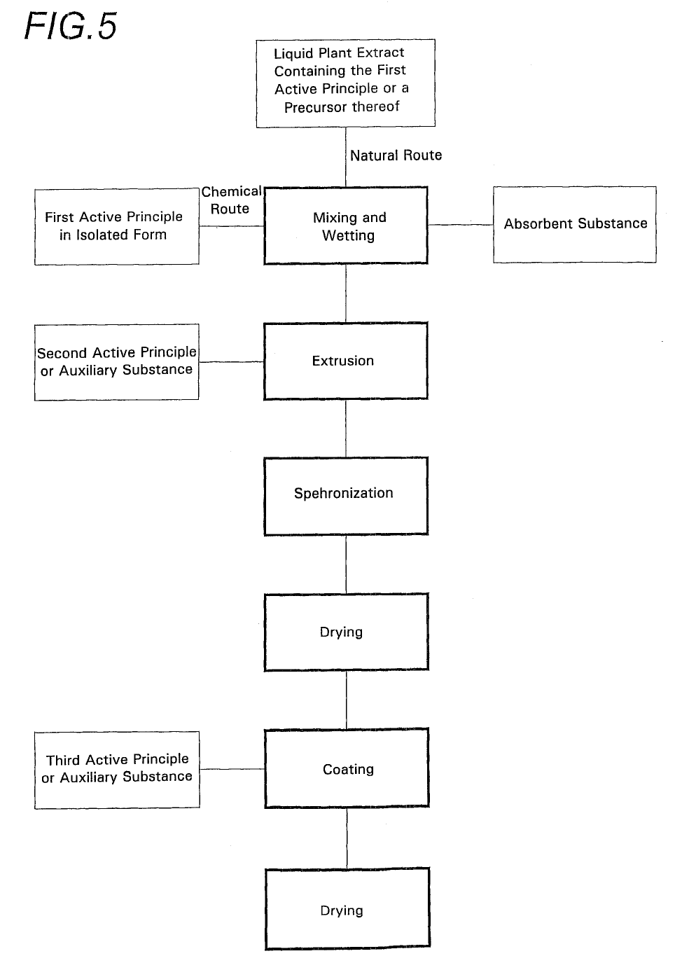

It is quite obvious that the coating step or steps according to the third embodiment of FIG. 4 may be combined with the introduction of an auxiliary substance or an active principle during extrusion according to the second embodiment illustrated in FIG. 3. A fourth embodiment combining all these steps has been shown in FIG. 5.

Advantageously, the method according to the invention allows production of a solid galenic form, that is easy to use and specifically capable of oral administration, without resorting to the use of elevated temperatures that can alter the fragile active principles originating from vegetables. Moreover, this galenic form ensures the physical and chemical stability over time of the active principles used, even under unfavorable storage conditions.

Surprisingly, the formulation, according to the invention, assists with strengthening the activity of the active principle, thereby producing more effective or comparably effective compounds, but with lower doses that are therefore less toxic to the organism.

This increased activity may be explained partially by the exceptional bio-availability of the active principles loaded on the spheroids according to the invention. Actually, after the oral absorption of the compound of the invention, rapid release of the active substances takes place within the stomach, with gastric liquid producing the necessary leaching. All the active principles are very quickly released in a controlled, reproducible manner.

Moreover, and in a manner essential to the invention, uniting the two sources of active principle in the core of a single composition achieves a synergy that increases the effectiveness of the active principle.

The active solution of vegetable origin, besides furnishing a supplementary quantity of active principle, also brings to the composition of the invention all the other compounds present within the plant accompanying the active principle or its precursor in the natural state. The active principle, thus enriched by the compounds initially present alongside it within the plant, has its efficacy reinforced through an actual synergistic effect.

For all these reasons, the spheroids of the invention constitute an original formulation and one of great interest for preventive and curative medicine.

The present invention will be better understood by reading the following examples.

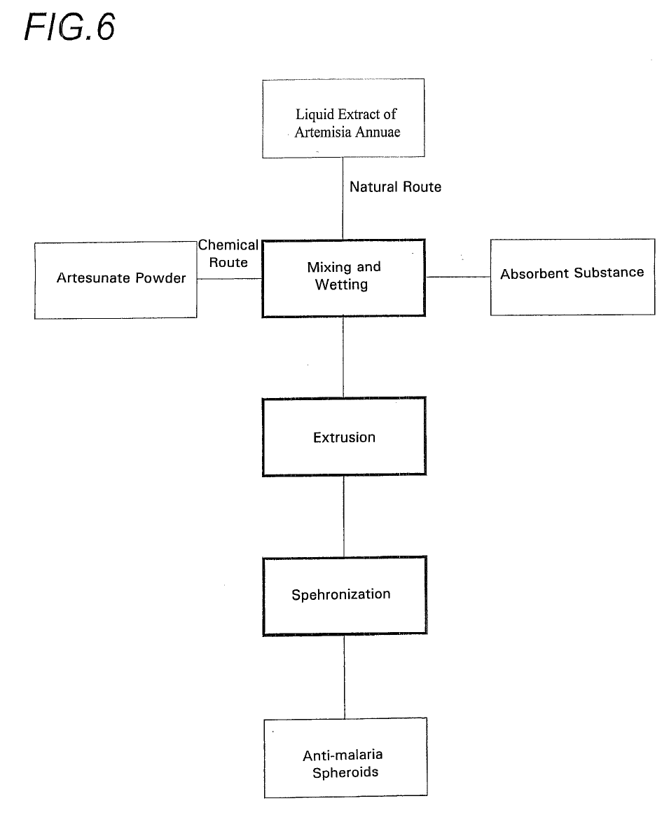

The first example has been shown in FIG. 6 and concerns a preferred application of the invention for the treatment of malaria.

The active principle used is the artesunate molecule, for example in the form of sodium salt. This active principle originates from the plant Artemisia Annua where it is found in the form of a precursor. To improve its stability and solubility, this precursor is next chemically transformed into dihydroartemisinine and then into artesunate.

In order to produce this exemplary application of the method according to the invention, a commercially available artesunate powder was used as the primary source of active principle in its isolated form.

A liquid extract, preferably a mother tincture, was obtained from Artemisia Annua plants by extraction in alcohol and constitutes the second source of active principle, in this case in the form of its precursor, following a natural route.

The artesunate powder and the mother tincture of Artemisia Annua were mixed with an absorbent and adsorbent substance, for example, powdered microcrystalline cellulose in conformance with the first step of the method according to the invention.

The Artemisia Annua mother tincture, serving as the wetting liquid, by mixing with the microcrystalline cellulose confers the necessary plastic properties for the remainder of the procedure.

Once it has been suitably wetted, the loaded wet substance was extruded and then spheronized according to the second and third steps of the invention in order to produce anti-malarial spheroids.

These artesunate-based spheroids made in accordance with the invention have proven to be surprisingly effective.

Actually the World Health Organization recommendation for the treatment of malaria is a utility dose of 200 mg (3.09 fr) of artesunate twice a day for three days. Using the spheroids of the invention, most clinical tests have shown that with such a dose, the parasite disappears from the blood in only 48 hours.

While retaining the treatment outline advocated by the WHO of two doses per day for three days, with the invention it is possible to achieve spheroids grouped for effective unitary administration containing dosages less than or equal to 150 mg (2.32 gr) of artesunate per administration for an adult. If clinical trials confirm this, it is hoped that spheroids can be produced that are sufficiently effective when packaged in amounts of about 120 mg (1.85 gr) of artesunate for each dose.

It is therefore possible to reduce the toxicity of treatment, but especially to reduce its price considerably, a crucial issue in countries struck by this illness.

Compositions containing lower doses are also envisioned, destined for use with children and preferably packaged in doses containing a quantity less than or equal to 100 mg (1.54 gr) of artesunate per dose.

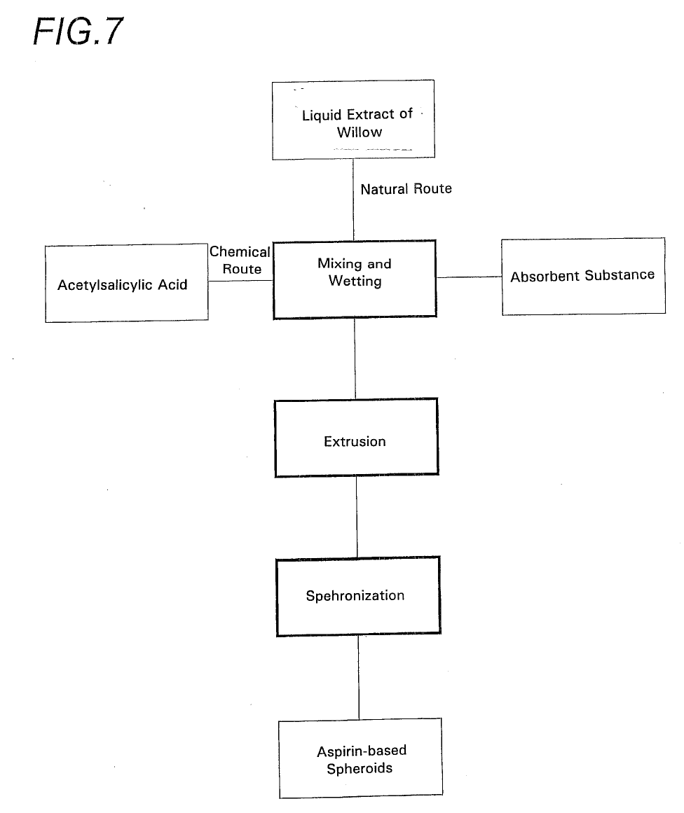

A second exemplary application of the invention is shown in FIG. 7 and concerns the formation of aspirin-based spheroids.

In this example a commercial acetylsalicylic acid powder, usually called aspirin, is used as the active principle originating from the first route called “chemical.”

The natural route entails by the use of liquid willow extract which contains the salicylic acid, precursor of acetylsalicylic acid. The liquid willow extract is preferably a mother tincture of willow obtained through hydro-alcohol extraction or other appropriate means.

According to the invention, first the two sources of active principle are mixed with an absorbent and adsorbent substance, for example, microcrystalline cellulose. The solid mass, wetted with liquid extract, is then extruded and next spheronized to obtain aspirin based spheroids in accordance with the invention.

When it is administered in a conventional galenic form and in the usual amount generally corresponding to an adult dosage of 1 g (15.43 gr) of acetylsalicylic acid per dose, aspirin is known to produce unwanted secondary effects, specifically, stomach and digestive system inflammation.

The invention permits the production of aspirin based spheroids that are satisfactory and effective with a significantly lower dose of acetylsalicylic acid. These spheroids can thus be packaged in units containing a dose generally equal to 0.1 g (1.54 gr) of acetylsalicylic acid per dose, for example.

Since the amount of active principle is greatly reduced, the toxicity of the composition is advantageously attenuated, thereby diminishing and in certain cases even eliminating harmful secondary effects.

It is apparent that the invention is not limited to the applications and preferred examples described above and shown in the different drawings, since a person skilled in the art may modify the invention, envision other variations, and conceive of numerous other applications without departing from the scope and context of the invention.

1-23. (canceled)

24. A method of producing spheroids, the method comprising the steps of:
furnishing a first active principle of vegetable origin in an isolated form;
furnishing a solution of vegetable origin comprising one of the first active principle and a precursor of the first active principle;
furnishing an absorbent having plastic properties compatible with the method;
mixing the first active principle of vegetable origin in the isolated form, the solution of vegetable origin comprising one of the first active principle and the precursor of the first active principle, and the absorbent having plastic properties compatible with the method;
wetting the mixture with a wetting liquid, if necessary;
extruding the wet mixture; and
spheronizing the extruded mixture to fabric the spheroids.

25. The method of producing spheroids according to claim 24, further comprising the step of, during the extrusion step, introducing at least one of another active principle and at least one auxiliary substance.

26. The method of producing spheroids according to claim 24, further comprising the step of, after the spheronizing step, drying spheroids.

27. The method of producing spheroids according to claim 24, further comprising the step of, after the spheronizing step, coating the spheroids with at least one coat of a second active principle, different from the first active principle of vegetable origin.

28. The method of producing spheroids according to claim 24, further comprising the step of, after the spheronizing step, separating the spheroids into spheroids which have specific sizes.

29. The method of producing spheroids according to claim 24, further comprising the step of, after the spheronizing step, covering the spheroids with a film.

30. The method of producing spheroids according to claim 24, further comprising the step of, after the spheronizing step, enclosing the spheroids in at least one of a casing and a capsule.

31. The method of producing spheroids according to claim 24, further comprising the step of chemically transforming the first active principle of vegetable origin from a molecule originating directly from a plant.

32. The method of producing spheroids according to claim 24, further comprising the step of furnishing the first active principle of vegetable origin in an isolated commercial form.

33. The method of producing spheroids according to claim 24, further comprising the step of furnishing, as the solution of vegetable origin, a liquid vegetable plant extract from which the at least one of the first active principle and the precursor of the first active principle is extracted.

34. The method of producing spheroids according to claim 33, further comprising the step of furnishing, as the solution of vegetable origin, a mother tincture of a plant from which the at least one of the first active principle and the precursor of the first active principle is extracted.

35. The method of producing spheroids according to claim 24, further comprising the step of furnishing, as the absorbent, an adsorbent comprising microcrystalline cellulose.

36. The method of producing spheroids according to claim 24, further comprising the step of furnishing, as the absorbent, a dry homogeneous mixture of a slightly substituted hydroxypropylated cellulose polymer.

37. The method of producing spheroids according to claim 24, further comprising the step of, during the mixing step, adding at least one auxiliary substance.

38. The method of producing spheroids according to claim 24, further comprising the step of utilizing the solution of vegetable origin as the wetting liquid for the wetting step.

39. A plurality of spheroids containing a first active principle of vegetable origin, the plurality of spheroids being prepared by a method comprising the steps of:
furnishing a first active principle of vegetable origin in an isolated form;
furnishing a solution of vegetable origin comprising one of the first active principle and a precursor of the first active principle;
furnishing an absorbent having plastic properties compatible with the method;
mixing the first active principle of vegetable origin in the isolated form, the solution of vegetable origin comprising one of the first active principle and the precursor of the first active principle, and the absorbent having the plastic properties;
wetting the mixture with a wetting liquid, if necessary;
extruding the wet mixture; and
spheronizing the extruded mixture to form the spheroids.

40. The plurality of spheroids according to claim 39, wherein the plurality of spheroids contain, on the one hand, at least an isolated active principle of vegetable origin and, on another hand, a solution of vegetable origin containing the active principle or a precursor thereof, absorbed and/or adsorbed on an absorbent and/or adsorbent substance.

41. The plurality of spheroids according to claim 39, wherein the plurality of spheroids are coated with at least one layer containing at least one second active principle.

42. The plurality of spheroids according to claim 39, wherein the first active principle of vegetable origin is artesunate and the solution of vegetable origin is a liquid extract of Artemisia Annua.

43. The plurality of spheroids according to claim 42, wherein the plurality of spheroids are grouped into unit doses of at least one of an adult dose equal to or smaller than 150 mg of artesunate and a child dose equal to or smaller than 100 mg of artesunate.

44. The plurality of spheroids according to claim 39, wherein the first active isolated principle of vegetable origin is acetylsalicylic acid and the solution of vegetable origin is liquid willow extract.

45. The plurality of spheroids according to claim 44, wherein the plurality of spheroids are grouped in unit doses of essentially equal to 0.1 g of acetylsalicylic acid.

46. The plurality of spheroids according to claim 39, wherein the first active principal of vegetable origin is a medicinal composition and the plurality of spheroids have a galenic form.
